(19) United States
(12) Patent Application Publication
(10) Pub. No.: US 2012/0197246 A1
(43) Pub. Date: Aug. 2, 2012

(54) ABLATION CATHETER

(75) Inventor: Kevin MAUCH, Windsor, CA (US)

(73) Assignee: Medtronic Vascular, Inc., Santa Rosa, CA (US)

(21) Appl. No.: 13/357,488

(22) Filed: Jan. 24, 2012

Related U.S. Application Data
(60) Provisional application No. 61/572,290, filed on Jan. 28, 2011.

(51) Int. Cl. A61B 18/18; A61B 18/14

(52) U.S. Cl. 606/33; 606/41

(57) ABSTRACT

An ablation catheter system including a radio frequency generator and an elongate catheter having an ablation element at the distal portion thereof The ablation element has at least one electrode electrically connected to the radio frequency generator and a shape memory component formed from a shape memory material. The shape memory component transforms the ablation element between a first straightened delivery configuration and a second deployed configuration. Thermal energy transfer between the electrode and the shape memory component transforms the shape memory component into the deployed configuration and places the electrode of the ablation element into contact with tissue at a treatment site. The transformation temperature of the shape memory material is a temperature above body temperature such that the transformation of the shape memory component is not activated by mere placement within the body but rather is activated by heat transfer from the electrodes.

This application claims benefit of U.S. Provisional Application No. 61/572,290, filed Jan. 28, 2011. The instant application claims the benefit of the listed application, which is hereby incorporated by reference herein in its entirety, including the drawings.

### FIELD OF THE INVENTION

The invention relates in general to a catheter, and more specifically to an ablation catheter.

### BACKGROUND OF THE INVENTION

Tissue ablation is used in numerous medical procedures to treat a patient. For example, ablation may be utilized to remove tissue as a treatment in cancer or to modify tissue as a treatment to stop electrical propagation through the tissue in patients with an arrhythmia. Often ablation is performed by passing energy, such as electrical energy, through one or more electrodes causing the tissue in contact with the electrodes to heat up to an ablative temperature.

Mammalian organ function typically occurs through the transmission of electrical impulses from one tissue to another. A disturbance of such electrical transmission may lead to organ malfunction. One particular area where electrical impulse transmission is critical for proper organ function is in the heart. Normal sinus rhythm of the heart begins with the sinus node generating an electrical impulse that is propagated uniformly across the right and left atria to the atrioventricular node. Atrial contraction leads to the pumping of blood into the ventricles in a manner synchronous with the pulse.

Atrial fibrillation refers to a type of cardiac arrhythmia where there is disorganized electrical conduction in the atria causing rapid uncoordinated contractions that result in ineffective pumping of blood into the ventricle and a lack of synchrony. During atrial fibrillation, the atrioventricular node receives electrical impulses from numerous locations throughout the atria instead of only from the sinus node. This overwhelms the atrioventricular node into producing an irregular and rapid heartbeat. As a result, blood pools in the atria that increases a risk for blood clot formation. Various ablation techniques have been proposed to treat atrial fibrillation, including the Cox-Maze procedure, linear ablation of various regions of the atrium, and circumferential ablation of pulmonary vein ostia. Many ablation catheters include a super-elastic element at the distal end of the catheter and depend upon the elastic spring or superelastic properties of the material to transform between a smaller diameter delivery configuration and a larger diameter deployed configuration that contacts the vessel wall. The super-elastic element is often constrained by a delivery sheath or guide catheter during delivery to a treatment site, and the delivery sheath or guide catheter is proximally retracted in order to expose and thus deploy the super-elastic element. However, the ablation element must have the ability to achieve a very small diameter delivery configuration in order to avoid friction with the inner diameter of a delivery sheath or guide catheter. Accordingly, there is a need for an improved ablation element that can achieve a very small diameter delivery configuration for use in a low profile catheter to avoid friction with the inner diameter of a delivery sheath or guide catheter.

In addition, recently there has been development in the area of renal neuromodulation as a treatment of heart arrhythmia. Recent studies have suggested that kidneys may play a role in atrial fibrillation, as well as other heart arrhythmia or other cardio-renal diseases. For example, U.S. Patent Application Publication No. 2010/0174282 to Demarais, herein incorporated by reference in its entirety, discloses neuromodulation of renal nerves and/or other neural fibers, which contribute to renal neural functions, can directly or indirectly increase urine output, decrease plasma renin levels, decrease tissue (e.g., kidney) and/or urine catecholamines (e.g., norepinephrine), increase urinary sodium excretion, and/or control blood pressure. Furthermore, the neuromodulatory effects may reduce renal sympathetic nerve activity, which may reduce the load on the heart and/or may provide a systemic reduction in sympathetic tone to reduce the patient's susceptibility to heart arrhythmia, such as atrial fibrillation. However, intravascular access to target areas in the renal arteries often requires a lower profile catheter than that required for other ablation procedures. Accordingly, there is a need for an ablation element that can achieve a very small diameter delivery configuration for use in a low profile catheter that may be utilized in a renal neuromodulation procedure.

### BRIEF SUMMARY OF THE INVENTION

Embodiments hereof are directed to an ablation catheter system including a radio frequency generator and an elongate catheter having an ablation element at the distal end thereof. The ablation element has at least one electrode electrically connected to the radio frequency generator and a shape memory component formed from a shape memory material. The shape memory component is for transforming, the ablation element between a first delivery configuration, such as a low profile straightened form, and a second deployed configuration, such as a pre-shaped coiled form. Thermal energy transfer between the electrode and the shape memory material causes the shape memory component to assume the deployed configuration and thereby places the electrode of the ablation element into contact with tissue at a treatment site.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of an ablation catheter system, wherein an ablation element at the distal end thereof is shown in a delivery configuration.

FIG. 1A is a cross-sectional view taken along line A-A of FIG. 1.

FIG. 1B is a perspective view of a dual lumen sleeve that may be utilized to couple the distal end of the ablation element to the distal end of the ablation catheter system.

FIG. 2 is a side view of the distal end of the ablation catheter system shown in FIG. 1, wherein the ablation element is shown in a deployed configuration.

FIG. 2A is a cross-sectional view taken along line A-A of FIG. 2.

FIG. 3 is a flow chart of a method of ablating tissue, wherein the method utilizes the ablation catheter system of FIG. 1.

FIG. 4 is a side view of an ablation catheter system according to another embodiment hereof, wherein an ablation element at the distal end thereof includes a tubular shape memory component shown in a delivery configuration.

FIG. 4A is a cross-sectional view taken along line A-A of FIG. 4.

FIG. 5 is a side view of the distal end of the ablation catheter system shown in FIG. 4, wherein the ablation element is shown in a deployed configuration.

FIG. 6 is a perspective view of a helical shape memory component according to another embodiment hereof, wherein the helical shape memory component is shown in a deployed configuration.

FIG. 7 is a perspective view of a distal end of the ablation element according to another embodiment hereof.

### DETAILED DESCRIPTION OF THE INVENTION

Specific embodiments are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the renal, coronary, and carotid arteries, embodiments hereof may also be used in any other body passageways where deemed useful. Furthermore, there is no intention to be bound by any expressed or implied theory presented in the preceding technical field, background, brief summary or the following detailed description.

Embodiments hereof relate to an ablation catheter having an ablation element that includes a shape memory component and one or more electrodes disposed over the shape memory component. The shape memory component is preset or preformed into a spiral or other desired geometry for the intended application and then subsequently mechanically straightened for delivery into the vasculature. Once the ablation element is positioned within the vasculature as desired, thermal energy transfer between the electrode(s) and the shape memory component causes the ablation element to assume the preset or shape memory geometry of the shape memory component to thereby place the electrodes into contact with the vessel wall such that the electrodes may be utilized to ablate tissue.

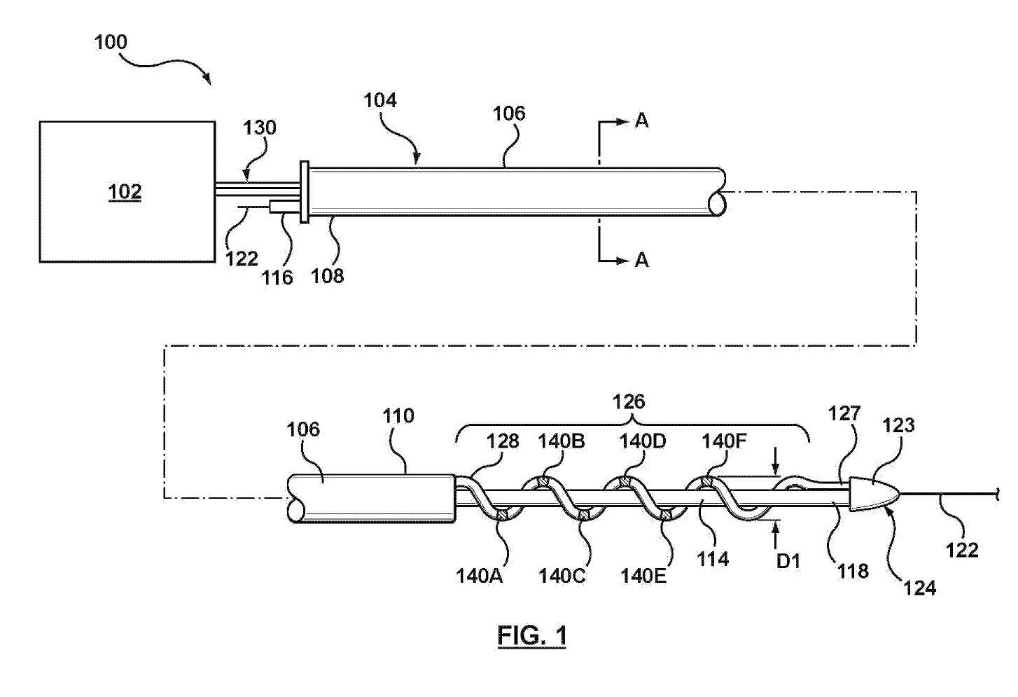

More particularly, referring to FIGS. 1 and 1A, an ablation catheter system 100 includes an external generator or power supply 102 and a catheter 104. Generator 102 supplies ablation energy to catheter 104. In an embodiment, generator 102 may be a multi-channel radio frequency generator such as the GENIUS™ generator produced by Medtronic Ablation Frontiers of Carlsbad, Calif. Catheter 104 includes an elongate flexible tubular outer shaft 106 defining at least one lumen 112 extending from a proximal end 108 to a distal end 110 thereof. In an embodiment, an outer diameter of outer shaft 106 is 0.065 inches or less. In the embodiment shown in FIGS. 1 and 1A, catheter 104 has an over-the-wire (OTW) catheter configuration with an inner or guidewire shaft 114 defining a guidewire lumen 120 that extends substantially the entire length of the catheter. Guidewire shaft 114 slidingly receives a guidewire 122 such that catheter 104 may be tracked over guidewire 122 in an over-the-wire manner. Guidewire shaft 114 is slidingly disposed within lumen 112 of outer shaft 106, and includes a proximal end 116 and a distal end 118. By “slidingly disposed,” it is meant that guidewire shaft 114 is allowed to slide relative to outer shaft 106. In one embodiment, guidewire shaft 114 is allowed to slide relative to outer shaft 106 via a thin-walled sleeve (not shown) that is attached to the inner surface of outer shaft 106 using thermal or adhesive bonding methods. The thin-wall sleeve may be formed out of a polymeric material such as but not limited to polyimide. Proximal ends 108, 116 of shafts 106, 114, respectively, extend out of the patient to be available for manipulation by a clinician, and distal ends 110, 118 of shafts 106, 114, respectively, are positionable at a target location within the vasculature.

Catheter 104 also includes an ablation element 126 that extends between distal end 110 of outer shaft 106 and distal end 118 of guidewire shaft 114. Ablation element 126 is positionable at a target location within the vasculature and includes at least one electrode for delivering ablation energy from generator 102 to a vessel wall. In the embodiment depicted in FIG. 1, ablation element 126 includes six electrodes 140A, 14013, 140C, 140D, 140E, 140F (collectively referred to herein as electrodes 140) equally spaced apart along the length of ablation element 126 although it will be apparent to one of ordinary skill in the art that the number of electrodes may be varied. Although only one electrode is required, multiple electrodes simultaneously ablate more surface area of tissue to result in a faster procedure time. In addition, it is not required that electrodes 140 be equally spaced apart but rather the distance between the electrodes may vary depending on the particular application. For example, the anatomy of the target treatment site within the vasculature may dictate the desired spacing of the electrodes, i.e., the distance between the electrodes as well as whether the electrodes are equally spaced apart or variably spaced apart. It will be understood by one of ordinary skill in the art that the length of ablation element 126 may vary according to its intended application. In an embodiment in which ablation element 126 is utilized in the renal arteries to perform a renal denervation or neuromodulation procedure, a longitudinal distance between first electrode 140A and last electrode 140F is typically between, but not limited to, 17 mm to 20 mm.

Electrodes 140 are preferably a series of separate band electrodes spaced along ablation element 126. Band or tubular electrodes are preferred because they have lower power requirements for ablation as compared to disc or flat electrodes, although disc or flat electrodes are also suitable for use herein. In another embodiment, electrodes having a spiral or coil shape may be utilized. In an embodiment, the length of each electrode 140 may range between 1-5 mm, and the spacing between each of electrode 140 may range between 1-10 mm. Electrodes 140 may be formed from any suitable metallic material including gold, platinum or a combination of platinum and iridium. In an embodiment, electrodes 140 are 99.95% pure gold. with an inner diameter of that ranges between 0.025 inches and 0.030 inches, and an outer diameter that ranges between 0.030 inches and 0.035 inches. Electrodes of smaller or larger dimensions, i.e., diameter and length, are also suitable for use herein.

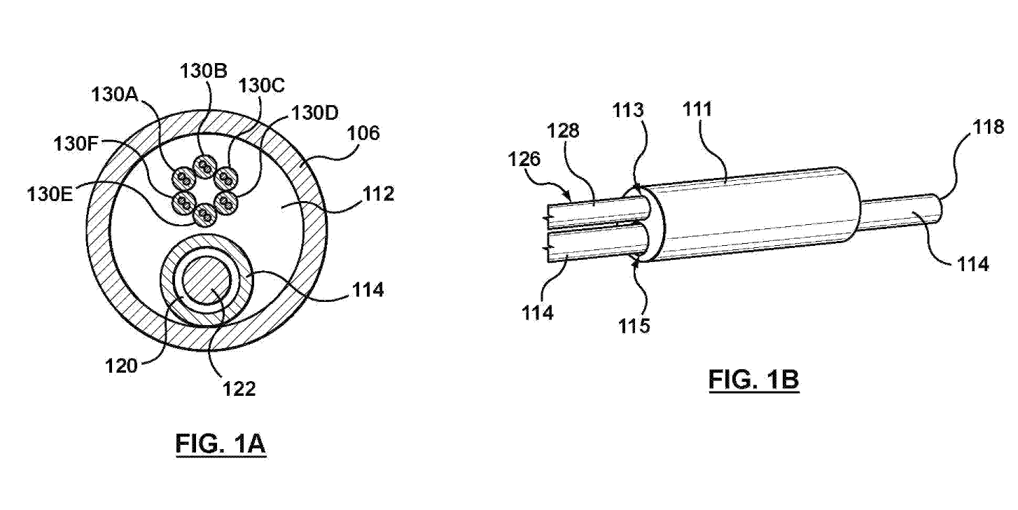

Each electrode 140 is electrically connected to generator 102 by a conductor or wire that extends through lumen 112 of outer shaft 106. The embodiment of FIG. 1 includes six electrodes 140A, 140B, 140C, 140D, 140E, 140F and six corresponding bifilar wires 130A, 130B, 130C, 130D, 130E, 130F (collectively referred to herein as wires 130) that electrically connect a respective electrode to generator 102. Each electrode 140 may be welded or otherwise electrically coupled to the distal end of its wire 130, as represented by connection 141 shown in FIG. 2A, and each wire 130 extends through outer shaft 106 for the entire length of catheter 104 such that a proximal end thereof is coupled to generator 102. In one embodiment, connection 141 is made between the distal end of a wire 130 and an inner surface of electrode 140.

With reference to FIG. 2A, in one embodiment, each wire 130 is a bifilar wire that includes a first conductor 132, a second conductor 134, and insulation 136 surrounding each conductor to electrically isolate them from each other. In an embodiment, first conductor 132 may be a copper conductor, second conductor 134 may be a copper/nickel conductor, and insulation 136 may be polyimide insulation. When coupled to an electrode, the two conductors bifilar wire 130 function to provide power to its respective electrodes and act as a T-type thermocouple for the purposes of measuring the temperature of the electrode. Temperature measurement provides feedback to generator 102 such that the power delivered to each electrode can be automatically adjusted by the generator to achieve a target temperature, and also provides an indication of the quality of the contact between the electrode and the adjacent tissue. For example, failure to reach the target temperature of 60° C. when high power such as greater than 8 W is being delivered suggests that the electrode is not making good tissue contact and may reside mainly in the bloodstream. Conversely, low power such as less than 2 W with target temperature achieved suggests that the electrode may be “buried” within the tissue wall and therefore may not experience adequate cooling from surrounding blood flow. In one embodiment, during the ablation procedure generator 102 may display the power each electrode 140 is receiving and the temperature achieved such that the user may assess each electrode's tissue contact.

In another embodiment hereof, wires 130 may be single conductor wires rather than the bifilar wires described above. Each single conductor wire provides power to its respective electrode but would not measure temperature of the electrode.

Ablation element 126 also includes a shape memory component 138 (see FIG. 2A) that extends at least the length of ablation element 126 and runs substantially parallel with bifilar wires 130. Shape memory component 138 is utilized to deploy or transform ablation element 126 from an unexpanded or delivery configuration shown in FIG. 1, i.e., a substantially straightened form, to an expanded or deployed configuration shown in FIG. 2, i.e., a preset spiral or helical form. More particularly, shape memory component 138 is constructed from a shape memory material that is pre-formed or pre-shaped into the deployed configuration, which has a specific geometry such as the spiral or helix shown in FIG. 2. Certain shape memory materials have the ability to return to a predefined or predetermined shape when subjected to certain thermal conditions. When shape memory materials, such as nickel-titanium (Nitinol) or shape memory polymers, are at a relatively low temperature, items formed therefrom may generally be deformed quite easily into a new shape that they retain until exposed to a relatively higher transformation temperature, which in embodiments hereof is above a normal body temperature of 37° C., that then returns the items to the predefined or predetermined shape they held prior to the deformation. Shape memory component 138 is formed from such a shape memory material to be inserted into the body in a deformed, low profile straightened state and to return to a “remembered” preset shape once shape memory component 138 is exposed to a transformation temperature in vivo. Thus, shape memory component 138 has at least two stages of size or shape, a generally straightened or stretched-out coil configuration of a sufficiently low profile for delivery to the treatment site as shown in FIG. 1 and a spiral or helical configuration that places electrodes 140 into contact with a vessel wall 201, which is shown as a dashed line in FIG. 2. The delivery configuration may be achieved by mechanical straightening shape memory component 138 by the operator, or by a tensioning device. Referring to FIG. 1, in an embodiment, a delivery diameter D1 of shape memory component 138 is between 1 and 2 mm to accommodate delivery to a target vessel such as a renal artery.

Ablation element 126 also includes an insulating component 128 which functions to electrically isolate shape memory component 138 from electrodes 140. Insulating component 128 is a tubular sheath defining a lumen 129 that is formed from an electrically insulative material, such as PEBAX. In an embodiment, insulating component 128 may have an outer diameter of approximately 0.027 inches and an inner diameter of approximately 0.023 inches. Insulating component 128 houses shape memory component 138 as well as houses wires 130 to provide additional protection thereto, and electrodes 140 are attached to or disposed around insulating component 128. A distal end 127 of insulating component 128 is attached to distal end 118 of guidewire shaft 114 by any suitable method such as an adhesive, a sleeve, or other mechanical method. In one embodiment depicted in FIG. 1, distal end 127 of insulating component 128 is attached to distal end 118 of guidewire shaft 114 via a cyanoacrylate adhesive and a polymer sleeve 123 surrounds and holds together the distal ends 127, 118 to form a tapered distal tip 124 of catheter 104.

As shown in the embodiment of FIG. 1 and FIG, 2, both shape memory component 138 and insulating component 128 extend along the length of ablation element 126 and proximally extend into distal end 110 of outer shaft 106 at least one or two centimeters such that the proximal end of shape memory component 138 is sufficiently removed from electrodes 140 to avoid any thermal effects therefrom. The proximal end of insulating component 128 may be attached to an inner surface of outer shaft 106 using thermal or adhesive bonding methods to have a fixed longitudinal position relative thereto, and the proximal end of shape memory component 138 may then be secured to an inner surface of insulating component 128. In another embodiment (not shown), both insulating component 128 and shape memory component 138 are elongated such that they extend the entire length of catheter 104. Insulating component extends through lumen 112 of outer shaft 106 and is attached to an inner surface of outer shaft 106 using thermal or adhesive bonding methods to have a fixed longitudinal position relative thereto. For the entire length of catheter 104, wires 130 and elongated shape memory component 128 are housed within lumen 129 of insulating component 128. In yet another embodiment (not shown), insulating component 128 includes a series of individual insulating bands or sleeves that are positioned inside of each electrode 140 to isolate the electrodes from shape memory component 138.

The transformation temperature of shape memory component 138 is set at a temperature that is just above body temperature, such as between 40 degrees C. and 45 degrees C. As shown in the cross-sectional view of FIG. 2A, shape memory component 138 is positioned adjacent to or abutting against an inner surface of insulating component 128 and band electrode 140 is disposed around and encircles insulating component 128. During an ablation procedure, generator 102 supplies power to electrodes 140 in order to heat electrodes 140 to a target temperature of between 60 degrees C. and 80 degrees C., which is suitable to ablate tissue. As electrodes 140 are heated to the target temperature, shape memory component 138 assumes the deployed configuration via thermal energy transferred from electrodes 140. In turn, shape memory component 138 deploys electrodes 140 of ablation element 126 into contact with vessel wall 201. As shape memory 138 assumes the deployed component, distal end 127 of insulating component 128 proximally retracts such that ablation element 126 radially expands into contact with vessel wall 201. Since distal end 127 of insulating component 128 is coupled to distal end. 118 of guidewire shaft 114, guidewire shaft 114 also slightly proximally retracts within outer shaft 106 in order to allow deployment of ablation element 126. In an embodiment, insulating component 128 may be formed from a thermoplastic material such as PEBAX, PEEK, polyimide, or nylon having ceramic filler mixed therein order to increase heat transfer between electrodes 140 and shape memory component 138. The ceramic filler for improving thermal conductivity of insulating component 128 may be, for example, aluminum nitride and boron nitride. Notably, the transformation/deployment of shape memory component 138 is activated by heat transfer from electrodes 140 and not merely due to the change in temperature from being placed in vivo. Since the user controls the activation of generator 102, proper positioning of ablation element 126 prior to deployment of the ablation element may be achieved.

The use of a heat-activated shape memory material for deployment of ablation element 126 allows for a simpler catheter design that avoids the requirement of deployment components built into the catheter. In addition, the use of a heat-activated shape memory material for deployment of ablation element 126 permits a low profile straightened delivery configuration that minimizes friction with an inner surface of a delivery sheath or guide catheter, which is usually present with self-expanding superelastic devices. Further, the use of a shape memory material for deployment of ablation element 126 provides reliable positioning of electrodes 140 against the vessel wall.

In another embodiment hereof, the shape memory component 138 assumes its deployed configuration, thereby radially expanding ablation element 126, without need for guidewire shaft 114 to proximally retract within outer shaft 106. More particularly, as shown in FIG. 1B, guidewire shaft 114 is not required to be slidingly disposed within the outer shaft 106 (not shown in FIG. 1B) but rather a dual lumen sleeve 111 having a first lumen 113 extending therethrough and a second lumen 115 extending therethrough is utilized for coupling the distal end of insulating component 128 adjacent to distal end 118 of guidewire shaft 114. The distal end of insulating component 128 is disposed within first lumen 113 and bonded to an inner surface of dual lumen sleeve 111, and guidewire shaft 114 is slidingly disposed within second lumen 115. Dual lumen sleeve 111 slides proximally along guidewire shaft 114 during deployment of shape memory component 138 (obscured from view in FIG. 1B) which allows ablation element 126 to radially expand without need for guidewire shaft 114 to move within outer shaft 106 (not shown in FIG. 1B).

In an embodiment, the deployed configuration of shape memory component 138 is a spiral or helical configuration that defines a blood flow lumen through the open center of the helix. In the embodiment shown in FIG. 2, the deployed spiral configuration of shape memory component 138 includes three revolutions or loops 144A, 144B, 144C (collectively referred to herein as loops 144) and includes a pitch spacing 146 between consecutive loops. Pitch spacing 146 may range between 2 mm and 8 mm, in one embodiment, pitch spacing 146 is about 5 mm and a deployed diameter D2 of each loop 144 is between 4 to 8 mm to place electrodes 140 into apposition and contact against renal arteries.

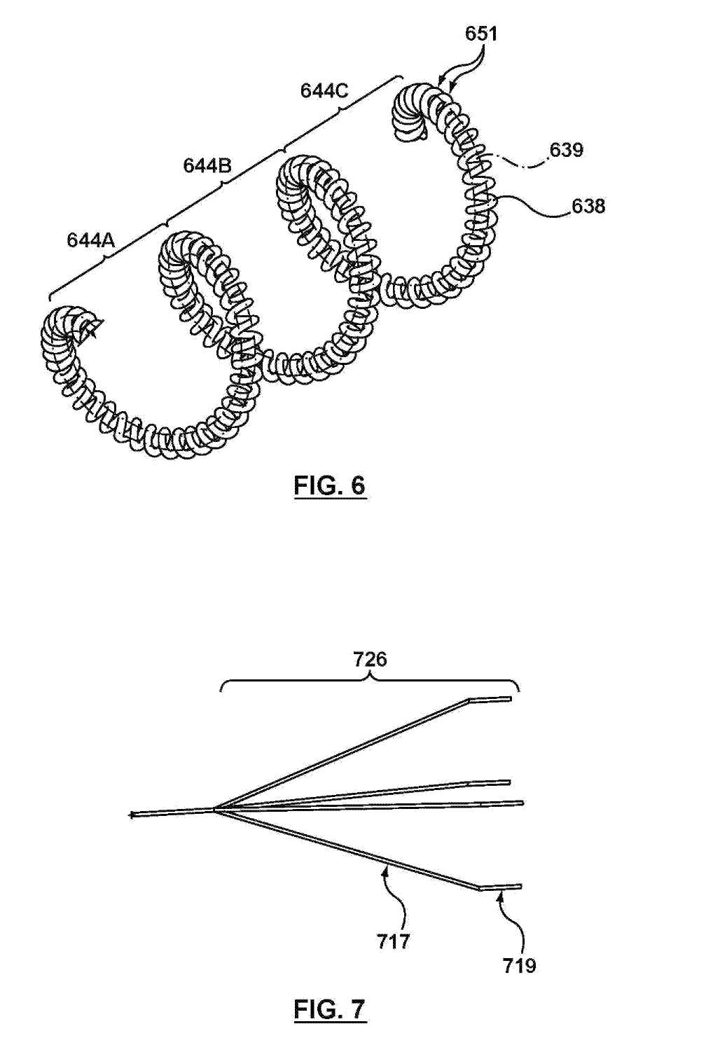

Although shown with a deployed configuration of a spiral or helix, it will be understood by one of ordinary skill in the art that ablation element 126 may have alternative deployed configurations for contacting the vessel wall. For example, ablation element 126 may form a single circumferential loop, formed in a plane transverse to the longitudinal axis of catheter 104, such as the configuration described in U.S. Pat. No. 6,773,433 to Stewart et al, and assigned to Medtronic, Inc., herein incorporated by reference in its entirety. In addition, the deployed configuration of ablation element 126 may have a radially increasing or decreasing helix such as the configuration described in U.S. Patent Application Publication No. 2004/0049181 to Stewart et al. and assigned to Medtronic, Inc., herein incorporated by reference in its entirety. Further, although ablation element 126 is shown as wound around the inner guidewire shaft 114 in FIG. 1, it will be understood by those of ordinary skill in the art that ablation element 126 may alternatively extend or be positioned longitudinally adjacent to and/or abutting against inner guidewire shaft 114. In another embodiment, the deployed configuration of ablation element 126 may form a basket or stent-like geometry, such as those described in U.S. Pat. No. 7,850,685 to Kunis et al. and assigned to Medtronic Ablation Frontiers LLC, herein incorporated by reference in its entirety. In another embodiment depicted in FIG. 7, the deployed configuration of an ablation element 726 may include a plurality of tines or fingers 717 that each deploy radially outward into apposition with a vessel wall. Longitudinal extensions 719 distally extend from the distal ends of tines 717 for abutting against the vessel wall. The electrodes (not shown in FIG. 7) of ablation element 726 are located on longitudinal extensions 719 for contacting and ablating tissue.

In an embodiment, shape memory component 138 is a NiTi (nitinol) wire having a diameter between approximately 0.008 inches and 0.012 inches. Wires of smaller or larger diameter are also suitable for use herein. The nitinol wire may have a round or circular cross-section. In other embodiments, the nitinol wire may have an elliptical cross-section, a strip or ribbon-like form or any other suitable cross-sectional configuration. Shape memory component 138 may have a very thin insulating sleeve 142 placed thereover to electrically isolate shape memory component 138 from the conductive bifilar wires 130. In one embodiment, insulating sleeve 142 is a layer of PET heat shrink having a wall thickness of approximately 0.0005 inches. Although embodiments described herein are not limited hereto, nickel-titanium or nitinol alloys suitable for use herein are described in fixed designation F2063, which states the standard material composition requirements for nickel-titanium shape memory alloys used in medical devices and surgical implants. Nitinol's properties, including transformation temperature, can vary with composition, thermo-mechanical processing, and finished component processing. Thus, as will be understood by those of ordinary skill in the art, varying the concentration of elements of NiTi and/or subjecting the formulation to one or more heat treating processing steps results in a material with a transformation temperature between 40° C. and 45° C. Nitinol is commercially available from several vendors, including NDC of Fremont, Calif., Memry of Bethel, Conn., and Fort Wayne Metals of Fort Wayne, Ind. During manufacture, a NiTi wire is placed into a shaping fixture made out of stainless steel or INCONEL™ which constrains and forms the NiTi wire into the desired shape. The assembly of the shaping fixture with NiTi wire therein is placed into a convection oven or salt pot at a temperature typically between 500° C. to 515° C. for a time of between 5 to 15 minutes. The assembly is then removed from the oven and quickly quenched in water to lock-in the desired shape memory configuration. Once the cooling is completed, the shaped NiTi wire is removed from the shaping fixture. The NiTi wire is soft and pliable at temperatures below the transformation temperature, enabling it to be deformed into the generally straightened delivery configuration described above. In another embodiment, as mentioned above, shape memory component 138 may be formed from a shape memory polymer. As will be understood by those of ordinary skill in the art, processing temperatures and times for heat setting a shape memory polymer may vary from those described above with respect to a NiTi wire. Examples of polymers that can be processed to exhibit shape memory characteristics include polyurethane, polyetheretherketone (PEEK), polymethylmethacrylate (PMMA), polyethylene terephthalate (PET) and polyethyteneoxide (PEO).

Catheter 104 may have any suitable working length, for example, 50 cm to 200 cm, suitable to extend to a target location within the vasculature. Catheter shafts 106, 114 can be of any suitable construction and made of any suitable material, such as an extruded shaft formed of any suitable flexible polymeric material. Non-exhaustive examples of polymeric materials for catheter shafts 106, 114 are HDPE, PEBAX, polyethylene terephalate (PET), PEEK, nylon, silicone, polyethylene, LDPE, HMWPE, polyurethane, polyimide, or combinations of any of these, either blended or co-extruded. In an embodiment, a proximal portion of outer shaft 106 may in some instances be formed from a reinforced polymeric tube, for example, as shown and described in U.S. Pat. No. 5,827,242 to Follmer et al., which is incorporated by reference herein in its entirety.

In the coaxial catheter construction of catheter 104, both wires 130 and guidewire shaft 114 extend through the entire length of outer shaft 106, substantially parallel to each other. Other types of catheter construction are also amendable to the invention, such as, without limitation thereto, a catheter shaft formed by multi-lumen profile extrusion (not shown). For example, the catheter outer shaft 106 may be of dual lumen construction with wires 130 extending through the first lumen thereof and guidewire shaft 114 extending through the second lumen thereof.

In another embodiment (not shown), catheter 104 may be modified to be of a rapid exchange (RX) catheter configuration without departing from the scope of the present invention such that guidewire shaft 114 extends within only a distal portion of catheter 104 for a length typically between 20 cm to 30 cm which facilitates use of a shorter guidewire, i.e., 180 cm in length, as opposed to a relatively longer guidewire, i.e., 300 cm in length, for the over-the-wire (OTW) configuration.

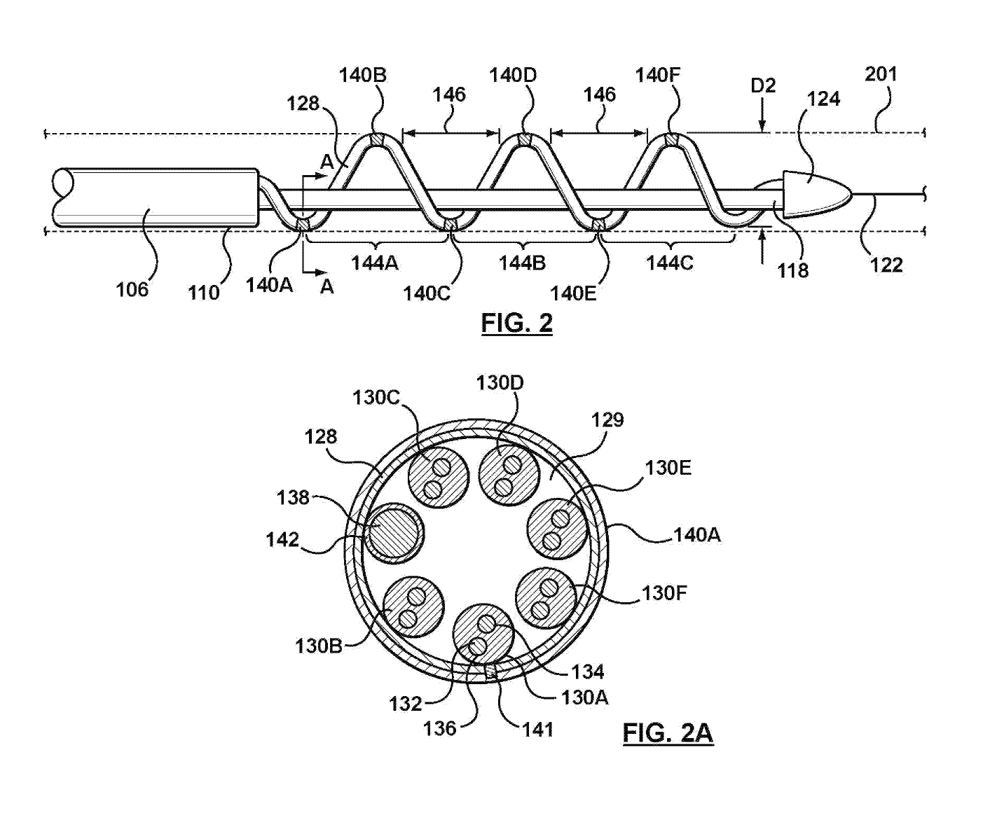

FIG. 3 depicts a flow chart illustrating the steps of a method of ablating tissue utilizing ablation catheter system 100 according to an embodiment hereof. In an embodiment, the tissue is nerve tissue in the renal arteries and ablation thereof is treatment for high blood pressure. However, it should be understood that the methods and apparatus described herein may be used for ablating tissue in other vascular applications and other body lumens. In step 350, catheter 104 is tracked through the vasculature to a treatment site. Prior to inserting catheter 104 into the vasculature, shape memory component 138 of ablation element 126 is substantially straightened into the delivery configuration of FIG. 1. Typically, a guiding catheter or sheath (not shown) is then inserted through an incision and into a femoral artery of a patient. Guidewire 122 may be advanced and navigated through the vasculature and catheter 104 may then be tracked thereover. During delivery, the guiding catheter may assist in maintaining shape memory component 138 and thus ablation element 126 in the straightened delivery configuration.

After ablation element 126 is positioned at the treatment site as desired, i.e., distal of the distal end of the guide catheter, ablation element 126 is deployed at step 352. More particularly, generator 102 is activated and the temperature of electrodes 140 begins to rise. As electrodes 140 are energized, heat transfer between electrodes 140 and shape memory component 138 occurs and shape memory component 138 assumes the deployed configuration, thereby deploying electrodes 140 of ablation element 126 into contact with the vessel wall. As previously described, the transformation temperature of shape memory component 138 is set at a temperature that is just above body temperature such that the transformation/deployment of shape memory component 138 is not activated by mere placement within the body but is rather activated by heat transfer from electrodes 140. During the ablation procedure, generator 102 may display the temperature achieved by each electrode such that the user is aware when the transformation temperature is reached. In addition, after deployment of shape memory component 138, the user may utilize fluoroscopic evaluation to visually confirm that radiopaque electrodes 140 are in contact with the tissue of the vessel wall.

After deployment of electrodes 140 into apposition with the vessel wall by shape memory component 138, generator 102 remains on and the temperature of electrodes 140 continue to rise until they reach a target temperature between 50 degrees C. and 80 degrees C. required to ablate tissue as shown in step 354. In one embodiment in which the tissue is nerve tissue in the renal arteries, electrodes 140 are heated to a temperature of 60° C. for a time period of between 20 to 240 seconds in order to ablate the target tissue. During the ablation procedure, generator 102 displays both the power supplied to each electrode as well as the temperature achieved by each electrode such that the user is aware when the electrodes reach the target temperature for ablation to occur.

Once ablation of the target tissue is complete, catheter 104 is removed from the vasculature in step 356. More particularly, generator 102 is turned off and blood flow within the vasculature cools electrodes 140 to body temperature. Shape memory component 138 is then straightened or otherwise compressed in order to enable removal thereof. In an embodiment, shape memory component 138 is straightened for removal by using the distal tip of the guide catheter (not shown). By proximally retracting catheter 104 into the guide catheter, or distally advancing the guide catheter over ablation element 126, the distal tip of the guide catheter compresses and/or straightens shape memory component 138 to a diameter sufficient to enable removal of ablation element 126. In another embodiment in which the distal end of insulating component 128 is fixed to guidewire shaft 114 and not slidable relative thereto, distal advancement of the guidewire shaft 114 may be utilized to stretch out or straighten shape memory component 138 into a lower profile for easier removal from the guide catheter. In another embodiment, a tensioning device (not shown) may be built into catheter 104 for mechanically straightening shape memory component 138 to enable removal of ablation element 126. For example, the distal end of guidewire shaft 114 may be tapered for use with a custom guidewire which has a solder ball or other means to create an interference fit at the distal end of the guidewire shaft. When the guidewire is advanced distally, the interference tit between the distal end of the guidewire shaft and the solder ball causes the distal end of the guidewire shaft to move distally, thus stretching out or straightening the shape memory component 138 into a lower profile. In another embodiment (not shown), ablation catheter system 400 also includes a slideable outer sheath that may be retracted and advanced over outer shaft 106. When the slidable outer sheath is distally advanced over ablation element 126, it acts to compress shape memory component 138 into a nearly straight configuration such that the entire ablation catheter system 400 may be removed from a guide catheter and the patient.

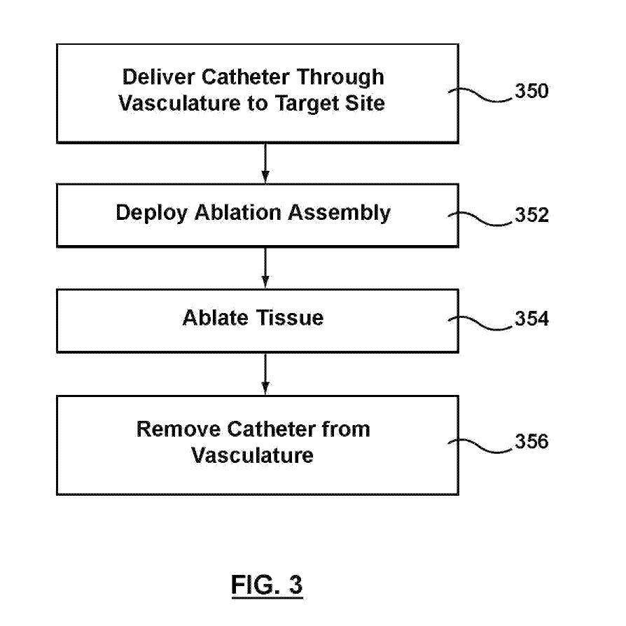

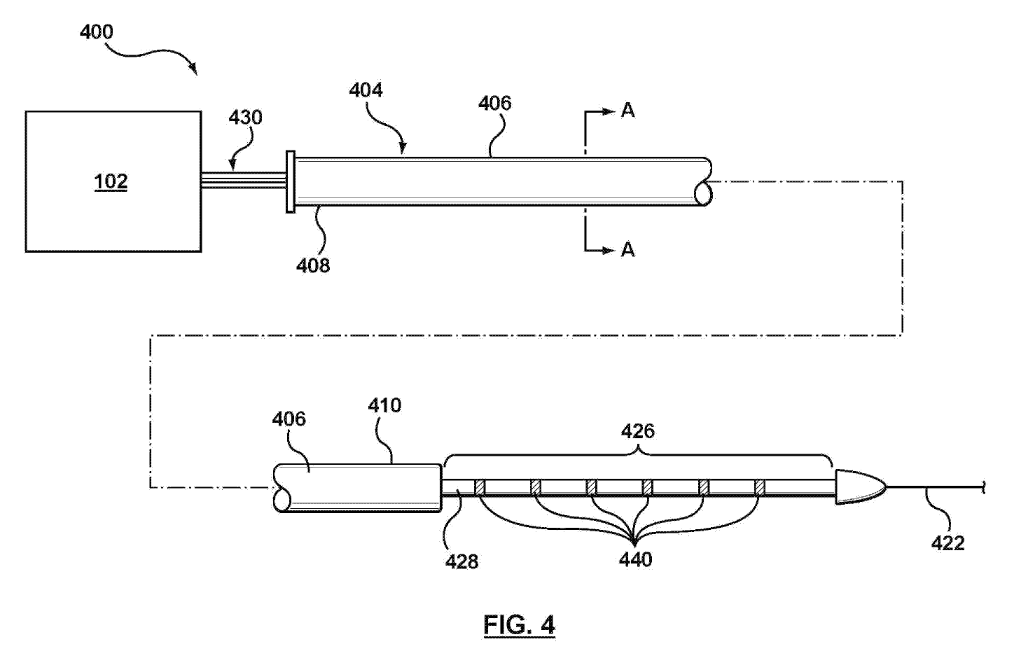

FIGS. 4, 4A, and 5 illustrate an ablation catheter system 400 according to another embodiment hereof. Ablation catheter system 400 includes an external generator or power supply 102 for supplying ablation energy to a catheter 404. Similar to catheter 104, catheter 404 includes an elongate flexible tubular outer shaft 406 defining at least one lumen 412 extending from a proximal end 408 to a distal end 410 thereof. Bifilar wires 430 and an elongated shape memory component 438 extends through the entire length of lumen 412 of outer shaft 406. At a distal end of catheter 404, electrodes 440 are disposed on an insulating component 428 and connected to bifilar wires 430 for receiving ablation power from generator 102. In one embodiment, catheter 404 does not include a separate inner guidewire shaft Rather, shape memory component 438 is a tubular construct formed from a shape memory material that defines a lumen 439 that accommodates a guidewire 422 such that catheter 404 may be tracked over guidewire 422 in an over-the-wire manner. As described above with respect to catheter 104, catheter 404 may be modified to be of a rapid exchange (RX) catheter configuration. The shape memory material of tubular shape memory component 438 may be metallic material such as NiTi (Nitinol) or a shape memory polymer. As described above with respect to system 100, thermal energy transfer between electrodes 440 and shape memory component 438 causes deployment of ablation element 426. The deployed configuration of ablation element 426 is shown in FIG. 5.

In addition to assisting in tracking catheter 404 to the treatment site, guidewire 422 may also be utilized for straightening tubular shape memory component 438. As described above, the shape memory component 438 must be substantially straightened to enable delivery of ablation element 426 to the treatment site and to enable retraction/removal of ablation element 426 after the ablation procedure is complete. Since shape memory component 438 is a pliable tube, guidewire 422 straightens out the predetermined shape thereof to allow for insertion into a guide catheter. Once ablation element 426 is positioned at the treatment site, guidewire 422 is proximally retracted within lumen 439 of shape memory component 438 until a distal end of guidewire 422 is located just proximal of ablation element 426. After the ablation procedure is complete, guidewire 422 may be distally advanced through lumen 439 of shape memory component 438, causing ablation element 426 to straighten out such that catheter 404 may be removed from the patient.

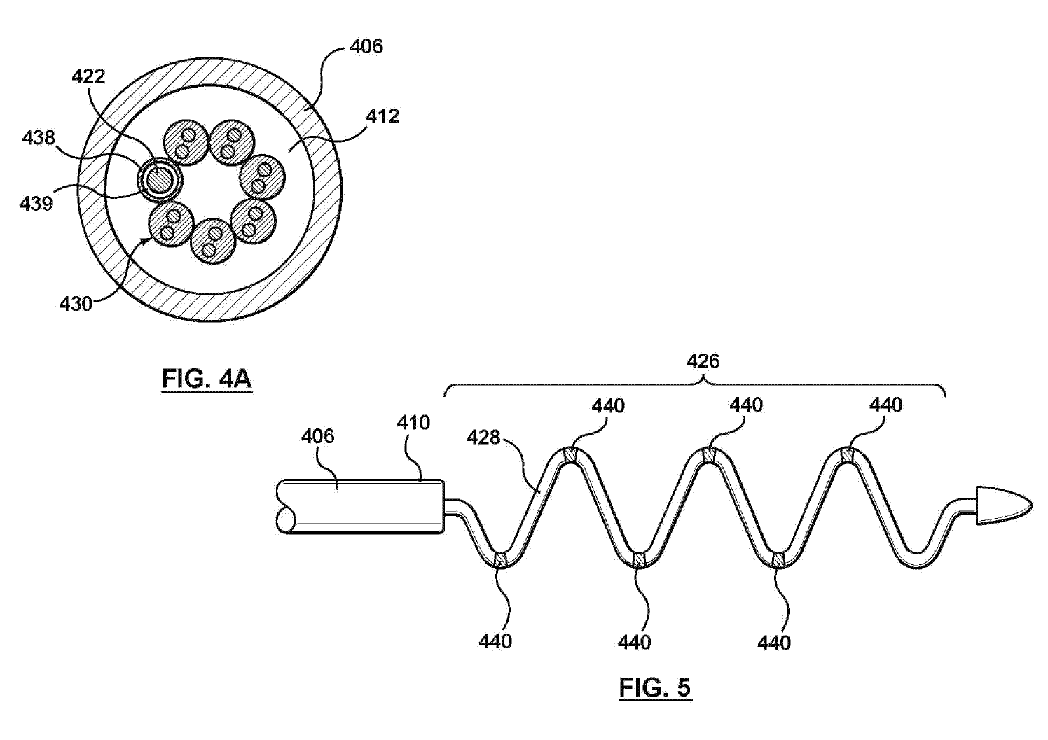

As an alternative to using a tubular shape memory component for receiving a guidewire, the shape memory component itself may be coiled into a helix having windings that define a lumen for accommodating a guidewire. More particularly, referring to FIG. 6, a distal portion of a shape memory component 638 is shown in its deployed configuration. Shape memory component 638 is an elongated solid or hollow wire-like component formed from material having shape memory characteristics such as NiTi (Nitinol) or a shape memory polymer. Shape memory component 638 is coiled into a helix having multiple windings 651 that define a lumen or passageway 639 to accommodate a guidewire (not shown), such as described above with respect to lumen 439 of tubular shape memory component 438. When shape memory component 638 assumes its deployed configuration, windings 651 coil into a helix or spiral that defines a blood flow lumen through the open center of the helix. Similar to the deployed configuration described above with respect to FIG. 2, the deployed spiral configuration of shape memory component 638 includes three revolutions or loops 644A, 644B, 644C. Thus, when deployed, shape memory component 638 has a “double helix” configuration.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An ablation catheter system comprising:
an energy source; and
a catheter having an ablation element disposed at a distal portion thereof, the ablation element including,
at least one electrode electrically connected to the energy source and
a shape memory component formed from a shape memory material, wherein thermal energy transfer between the at least one electrode and the shape memory component transforms the shape memory component and thereby the ablation element from a straightened delivery configuration to a deployed configuration for placing the at least one electrode of the ablation element into contact with tissue at a treatment site.

2. The ablation catheter of claim 1, wherein the catheter includes an outer shaft and an inner shaft and the ablation element extends between a distal end of the outer shaft and a distal end of the inner shaft.

3. The ablation catheter of claim 2, wherein a distal end of the ablation element is slidingly coupled to the distal end of the inner shaft via a dual lumen sleeve.

4. The ablation catheter of claim 1, wherein the ablation element further includes an insulating component disposed between the at least one electrode and the shape memory component to electrically isolate the at least one electrode from the shape memory component, the insulating component being formed of a material that allows the thermal energy transfer between the at least one electrode and the shape memory component.

5. The ablation catheter of claim 4, wherein the insulating component is formed from a thermoplastic material having ceramic filler mixed therein.

6. The ablation catheter of claim 1, wherein the at least one electrode is electrically connected to the energy source via at least one wire that has a proximal end coupled to the energy source and a distal end coupled to the electrode and wherein the at least one wire is a bifilar wire that includes a first copper conductor, a second copper or nickel conductor, and insulation surrounding each of the first and second conductors to electrically isolate them from each other.

7. The ablation catheter of claim 1, wherein the ablation element includes a series of band electrodes.

8. The ablation catheter of claim 1, wherein the deployed configuration of the shape memory component is a helix.

9. The ablation catheter of claim 1, wherein the shape memory material is nitinol.

10. The ablation catheter of claim 9, wherein the shape memory component is a solid wire covered by a thin layer of insulative material.

11. The ablation catheter of claim 1, wherein the shape memory component has a lumen therethrough sized to accommodate a guidewire.

12. The ablation catheter of claim 1, wherein the shape memory material is polymeric.

13. The ablation catheter of claim 1, wherein a shape transformation temperature of the shape memory component is just above body temperature at a temperature between 40 degrees C. and 45 degrees C.

14. A method of ablating tissue at a treatment site, the method comprising the steps of:
tracking a catheter through the vasculature to a treatment site, the catheter having an ablation element disposed at a distal portion thereof, the ablation element including at least one electrode electrically connected to an energy source and a shape memory component formed from a shape memory material, wherein the shape memory component is in a straightened delivery configuration;
positioning the ablation element at the treatment site;
supplying radio frequency energy to the at least one electrode from the energy source such that thermal energy transfer between the at least one electrode and the shape memory component transforms the shape memory component and thereby the ablation element into a deployed configuration that places the at least one electrode of the ablation element into contact with tissue at the treatment site; and
continuing to supply radio frequency energy to the at least one electrode until tissue at the treatment site is ablated.

15. The method of claim 14, wherein the deployed configuration is a helix.

16. The method of claim 14, further comprising the step of:
straightening the shape memory component and thereby the ablation element after ablation of tissue at the treatment site and
removing the catheter from the vasculature.

17. The method of claim 16, wherein the shape memory component has a lumen therethrough and the step of straightening the shape memory component includes distally advancing a guidewire into the lumen of the shape memory component.

18. The method of claim 14, wherein a shape transformation temperature of the shape memory component is just above body temperature at a temperature between 40 degrees C. and 45 degrees C. and the step of supplying radio frequency energy to the at least one electrode includes heating the shape memory component to the shape transformation temperature.

19. The method of claim 18, wherein the step of continuing to supply radio frequency energy to the at least one electrode includes heating the electrodes to a temperature between 60 degrees C. and 80 degrees C. In order to ablate tissue at the treatment site.

20. The method of claim 14, wherein the treatment site is nerve tissue in the renal arteries.
